(19) United States
(12) Patent Application Publication
(10) Pub. No.: US 2018/0180626 A1
(43) Pub. Date: Jun. 28, 2018

(54) A METHOD FOR DEVELOPMENT OF RECOMBINANT PROTEINS WITH FINGERPRINT LIKE SIMILARITY TO THE REFERENCE PRODUCT

(71) Applicant: STC Biologics, Inc., Cambridge, MA (US)

(72) Inventors: Magdalena Leszczyniecka, Cambridge, MA (US); Zahra Shahrokh, Cambridge, MA (US)

(21) Appl. No.: 15/322,857

(22) PCT Filed: Jul. 1, 2015
(86) PCT No.: PCT/US15/38888

§ 371 Date: Dec. 29, 2016

Related U.S. Application Data
(63) Continuation-in-part of application No. PCT/US15/11226, filed on Jan. 13, 2015.
(60) Provisional application No. 62/019,776, filed on Jul. 1, 2014.

(51) Int. Cl. G01N 33/68; C12P 21/00

(52) U.S. Cl. CPC G01N 33/6842; G01N 33/6848; C12P 21/00

(57) ABSTRACT

The present invention relates to the methods of developing recombinant proteins with a fingerprint like similarity to the reference product or the originator. The method is particularly useful in the development of biosimilar products. This method can also be used to establish comparability during the manufacturing process change for the originator products. Hie methods described herein are used to obtain a recipe for the production of a biosimilar product or a recombinant protein using a process that may be different from the original but that yields a recombinant protein that has fingerprint level of similarity to the reference product. The methods described herein can also used to obtain a fingerprinting analysis package for a biosimilar that can be submitted to regulatory agency for abbreviated biosimilar approval. While currently available analytical methods can identify and quantitate specific modifications on a recombinant, protein, no methods currently exist to measure and determine the concentration of product variants in a complex: mixture. The analytical methods described herein provide for identification and quantitation of the modifications of the recombinant proteins and of product variants in a complex mixture by utilizing various in silico computational approaches to transform analytical data and derive product variant distribution.

### FIELD OF THE INVENTION

The present invention relates to the methods (if developing recombinant proteins with a fingerprint like similarity to the reference product or the originator. The method is particularly useful in the development of biosimilar products. This method can also be used to establish comparability during the manufacturing process change for the originator products.

The methods described herein are used to obtain a recipe for the production of a biosimilar product or a recombinant protein using a process that may be different from the original but that yields a recombinant protein that has fingerprint level of similarity to the reference product. The methods described herein can also used to obtain a fingerprinting analysis package for a biosimilar that can be submitted to a regulatory agency for abbreviated biosimilar approval.

### BACKGROUND OF THE INVENTION

Recombinant proteins arc a major class of biologic drugs used to treat a wide range of diseases. They are called biologics as they are produced in living cells. Production of recombinant proteins in cells is complicated by the fact that a cell's host proteins can modify recombinant proteins by adding a variety of modifications to the product and making a product heterogeneous. This heterogeneity results in a recombinant protein product that is a complex mixture of different recombinant protein product variants, each variant characterized by having a different combination of modifications.

Biosimilars are copies of the originator recombinant proteins. They are called bio-similar and not bio-generic as they arc not identical to the originator; the term ‘generic’ implies structural identity. Biosimilars with a fingerprint level of similarity are copies of the originator recombinant proteins that are almost indistinguishable from the originator on the analytical level, and in some cases could be classified as bio-generic, or bio-identical.

A major reason for producing a recombinant protein with a fingerprint like similarity is to:
a. ensure same product safely and efficacy as the original product, the originator,
b. limit development cost to obtain market approval for a biosimilar product.

Thus far, producing indistinguishable biosimilar or a bio-generic has not been possible.

The methods described herein delineate how to produce recombinant proteins with a fingerprint level similarity to the reference product and how to produce biosimilars with a fingerprint similarity to products from third parties, such as originator products.

The methods described herein delineate the analytical methods for showing fingerprint level similarity of the biosimilar to a third party's product.

While the idea of fingerprinting has been described in Kozlowski et al., 2011, indicating that a rigorous “fingerprint” similarity could remove many of the uncertainties of the biosimilar product relative to the originator, thus far a method for “fingerprinting” has yet to be developed. The challenge with developing such a methodology is that biologics are complex mixtures of many product variants, where each variant may have a combination of different modifications. For example, different manufactured antibody lots produced even by the same company could have different modifications including but not limited to glycans, oxidized amino acids, aggregated forms, and C-terminal lysines. When all of these modifications are taken into account, there is the potential for tens of thousands of product variants within each lot, each with the possibility to influence biological activity to different degrees.

For purposes of this specification it is important to understand the difference between a product variant and a product modification that exists on a protein. While currently available analytical methods such as mass spectrometry, chromatography and others can identity and quantitate specific modifications on a recombinant protein, no methods currently exist to measure and determine the concentration of product variants in a complex mixture. Each product variant is composed of the recombinant protein with a specific subset of modifications and complex biologic mixtures are composed of many product variants.

Product modifications include but are not limited to glycosylation. carboxylation, deamidation, oxidation, hydroxylation, O-sulfation, amidation, glycylation, glycation, alkylation, acylation, acetylation, phosphorylation, biotinylation, formylation, lipidation, iodination, prenylation, oxidation, palmitoylation, phosphatidylinositolation, phosphopantetheinylation, sialylation, and selenoylation, C-terminal Lysine removal.

The analytical methods applicable to the present disclosure include those that are capable of identifying and/or quantitating the modifications present on recombinant proteins and then identifying and quantitating product variants in a complex mixture, some of which may utilize various in silico computational approaches using the analytical data as input to derive a product variant distribution.

The in silico computational approaches that may be used to identify product variants from the analytical data identifying and quantitating product modification data include but are not limited to simulation, neural networks and artificial intelligence.

To develop a biosimilar recombinant protein with a fingerprint level similarity, the distribution of product variants in biosimilar product lots must fit within the range of the distribution observed for all tested originator or reference product lots, which are likely to have slightly different product variant distributions.

If small differences in product variants are present in a biosimilar product as compared to the originator, these product variants can be assessed (or their biological activity using the fingerprinting platform described herein via structure-activity-relationship (SAR). While SAR is routinely established for small molecules, such methodology has not yet been developed for biologic products. In essence for a recombinant protein the SAR is defined by the relationship between a modification and its effect on biologic activity.

The computational approaches that may be used to establish SAR equation include hut are not limned to neural networks, multivariate analysis, Partial Least Squares Regression (PLSR), Principal Components Regression (PCR), artificial intelligence and machine learning.

To establish SAR for a said recombinant protein one has to understand the impact of the various modifications alone and in combination on the biological activity of said recombinant protein. In order to achieve this level of understanding, one has to produce the recombinant protein enriched for each modification and test those variants in biological assay to determine the impact. It is expected that:
a. some modifications will have no effect on biological activity,
b. other modifications Will have a profound effect on biological activity.
c. it is also anticipated that combinations of some modifications may have synergistic or additive effects on biological activity.

SAR is used to determine whether specific product variants may negatively or positively impact biological activity. These variants can then be varied in concentration or eliminated by changing production processes.

There are two ways to change the distribution of product variants of a complex mixture;
a. By altering cell culture process (upstream). Host cell proteins affecting specific modifications on recombinant protein are first identified and modulators necessary to modulate those host proteins are then selected. Host proteins include enzymes involved in glycosylation, carboxylation, hydroxylation, deamidation. oxidation, C-terminal sulfation, C-terminal carboxylase and amidation or any other posttranslational modification. Modifying the activity of these enzymes using small molecules, natural products, biologics, RNAi, RNA, or DNA can be used for production of a recombinant protein with target modifications. A method that is capable of altering modifications on recombinant proteins are preferred for use in the production of biosimilar and biobetter biologics than known systems that knock-out modifications altogether. This method can produce recombinant proteins within target ranges as opposed to knock out technologies which have no possibility of targeting a desired modification range.
b. During protein purification process (downstream) specific chromatography steps such affinity, ion exchange or mixed mode chromatogaphy are used to remove specific product variants. Examples include but are not limited to removal of specific glycosylation variants by lectin based chromatography, removal of certain charge variants such as deamidated and oxidized species by ion exchange and mixed-mode chromatography.

As with biosimilar development, the methodology described herein can be applied to other areas of biologic drug development. In particular, the disclosed methods have an application to situations where a production process for an originator biologic product needs to be changed. The key reason for a process change for originator recombinant proteins is to improve the cell line performance, to increase productivity and stability without changing modifications of said recombinant protein.

### SUMMARY OF THE INVENTION

The present invention provides methods for developing recombinant proteins with a fingerprint like similarity to reference products or originator products. The methods are particularly useful for biosimilar development. The method includes five components (A) analytical methods for measuring modifications on recombinant proteins (B) in vitro and in vivo assays to measure biological activity (C) methods used for recombinant protein variant and structure activity relationship determination (D) cell culture methods for optimization of cell culture conditions to produce the recombinant protein with the fingerprint level similarity to the originator and (E) purification methods to produce a recombinant proteins with the fingerprint level similarity to the originator.

Analytical methods for showing fingerprint similarity include chromatography methods to separate and quantitate different modifications as well as mass spectrometry methods to identify product modifications. The chromatography methods include but are not limited to size exclusion, ion exchange, reverse-phase, hydrophobic interaction chromatography, and released glycan analysis. Mass spectrometry methods including but are not limited to intact mass and reduced mass analysis, peptide map and disulfide linkage analysis.

Biological activity is intrinsic to each recombinant protein being optimized. Frequently used bioassays used to test biological activity include but are not limited to: target binding ELISA assay, binding to cells expressing receptor, receptor internalization, receptor phosphorylation assays as well as assays that measure functional activity such as proliferation assays.

Manufacturing methods focus on optimization of cell culture conditions via addition of modulators) to growth media containing living cells that produce recombinant proteins. Addition of modulator(s) to the living cell culture medium can be used to reduce or augment the activity of specific host protein(s) that control modifications on the recombinant protein, which may be a biosimilar. The modulators arc selected to modulate the activity of host proteins responsible for producing modifications. The modifications may include, but are not limited to, any of the following modifications: glycosylation, carboxylation, deamidation, oxidation, hydroxylation, O-sulfation, amidation, glycylation, glycation, alkylation, acylation, acetylation, phosphorylation, biotinylation, formylation, lipidation, iodination, prenylation, oxidation, palmitoylation, phosphatidylinositolation, phosphopantetheinylation, sialylation, and selenoylation, C-terminal Lysine removal.

Additional manufacturing methods can be used to obtain fingerprint like similarity on the recombinant protein being optimized. They include purification methodologies to remove undesired product species. Examples include but are not limited to removal of specific glycosylation variants by lectin-based chromatography, removal of deamidated and oxidized charge variants such as deamidated by ion exchange and mixed-mode chromatography.

The present invention provides methods to identify, quantify, remove, and assemble product variants to produce a biosimilar that exhibits fingerprint level of similarity to the originator.

In one aspect of the invention, there is provided a method for producing a biosimilar product showing a fingerprint level similarity to the originator;
a. Establishing a relationship between product modifications and biological activity;
   i. Identifying the number (n) of modifications present on a recombinant protein;
   ii. Preparing recombinant protein variants enriched for one or two modifications at the time, at least at three different levels (high, medium, low) for a total of 3n enriched variants produced;
   iii. Confirming the setoff modifications in the enriched population using HPLC and MS based assays;
   iv. Measuring biological activity of the enriched recombinant protein generated in ii), using biological assays relevant for said recombinant protein;
   v. Establishing a relationship between the modification and the biological activity;
b. Measuring the quantity and type of specific modifications found on at least three originator batches using analytical assays;
c. Setting target profile ranges for the modifications of the originator based on data generated in b).
d. Growing living cells expressing the biosimilar with the identical amino acid sequence to the originator;
e. Isolating the biosimilar from d) and comparing its modifications to the target set in b).
f. Selecting a plurality of growth media and one or more modulators to change modifications on the biosimilar and growing the cells in the presence of said modulators. Modulators can be selected from the library of modulators;
g. Isolating the product from f). and comparing its modifications to the target profile set in c);
h. Repeating steps f), g) with additional modulators and or at different modulator concentrations to match modifications set in c). The modulators can be used alone or in a combination with each other. The set of exact modulation required to obtain the target profile provides a recipe for the production of said biosimilar and cell culture conditions are established to obtain the target profile. The target profile should not be set outside the specifications set for said originator;
i. Once the cell culture production process is optimized, isolating the optimized product through a series of purifications steps which include but are not limited affinity, ion exchange or mixed mode chromatography with a goal to remove specific product variants;
j. Measuring the quantity and type of specific modifications found OH the biosimilar and comparing it to the target profile in c);
k. Determining product variants for each product batch using analytical data produced in b). and in j);
l. Comparing the type and quantity of the biosimilar product variants to the range of product variants produced by an originator;
m. Determining the impact of each product variant on biological activity based on the structure activity relationship and summing up the biological activity of all variants based on their relative abundance to identify whether the biological activity of the biosimilar is within the range for the biological activity the originator;
n. If specific product variants need to be removed, selecting a plurality of growth media and one or more modulators to change modifications on the biosimilar and growing the cells in the presence of said modulators. Modulators can be selected from the library of modulators. Isolating the product from n). through a series of purifications steps which include but are not limited to a affinity, ion exchange or mixed mode chromatography with a goal to remove specific product variants;
o. Confirming that biological activity of the biosimilar is within 80 to 125% of the originator in in vitro and in vivo biological assays;

In another aspect of the invention, there is provided a method for a process change for an originator with a fingerprint level similarity to the reference standard:
a. Establishing a relationship between product modifications and biological activity:
   i. Identifying the number (n) of modifications present on a recombinant protein;
   ii. Preparing recombinant protein variants enriched for one or two modifications at the time, at least at three different levels (high, medium, low) for a total of 3n enriched variants produced;
   iii. Confirming the identity of each enriched variant using HPLC and MS based assays;
   iv. Measuring biological activity for the recombinant protein variants generated in ii), using biological assays relevant for said recombinant protein;
   v. Establishing a relationship between the modification and the biological activity;
b. Measuring the quantity and type of specific modifications found on the reference product or alternatively using product specifications to set the target profile range;
c. Growing living cells expressing the originator product in the presence of growth media that produces higher titer or other beneficial cell line characteristics;
d. Selecting a plurality of one or more modulators to change modifications on the originator product produced using a new process and growing the cells in the presence of said modulators. Modulators can be selected from the library of modulators;
e. Isolating the product from d). and comparing its modifications to the target set in b);
f. Repeating steps d), e) with additional modulators and or at different modulator concentrations to match modifications set in b). The modulators can be used alone or in a combination with each other. The set of exact modulation required to obtain the target profile provides a recipe for the production of said comparable biologic. Target profile should not be set outride the Specifications set for said originator;
g. Once the cell culture production process is optimized, isolating the optimized product through n series of purifications steps which include but are not limited affinity, ion exchange or mixed mode chromatography with a goal to remove specific product variants;
h. Measuring the quantity and type of specific modifications found on the originator product produced using a new production process and comparing it to the target in b);
i. Determining product variants for each product batch using analytical data produced in b). for the reference product and in h). for the originator produced using a new production process.
j. Comparing the type and quantity of the originator product variants produced using new optimized process to the range of product variants produced by the original process;
k. Determining the impact of each product variant on biological activity based on the structure activity relationship; adding the biological activity of all variants based on their relative concentration to identify whether the theoretical biological activity of the originator produced using a new process is within the range for the original process;
l. If specific product variants need to be removed, selecting a plurality of growth media and one or more modulators to change modifications on the originator produced using the new process and growing the cells in the presence of said modulators. Modulators can be selected from the library of modulators; Isolating the product from n). isolating the optimized product through a series of purifications steps which include but are not limited affinity, ion exchange or mixed mode chromatography with u goal to remove specific product variants;
m. Confirming that biological activity of the originator produced using new process is within 80 to 125% of the originator produced using the original process;

The method for optimization may be used in conjunction with a bioreactor. shake flask or a wave bag or any other method known to one skilled in the art of process development. Assays selected for their ability to detect and measure the presence of specific modifications are used to measure modification. The assay module may be in liquid communication with the bioreactor for delivery of a recombinant protein to the assay module or can be carried out manually. The method can be implemented using a system having a library of individual modulators, which may be in liquid communication with the cell culture media and can be controlled by the assay module for transfer of individual modulators into the bioreactor, a shake flask or other cell culture container.

The foregoing summary and detailed description is better understood when read in conjunction with the accompanying drawings, which are included by way of example and not by way of limitation.

### BRIEF DESCRIPTION OF THE FIGURES

FIG. 1 contains the list of examples of host proteins and some of the known inhibitors.

FIG. 2 is a schematic representation of a glycosylation pathway.

FIG. 3 provides an example of a chromatogram showing the carbohydrate peaks using the 2AB method of carbohydrate analysis.

FIG. 4 schematic of an antibody showing different antibody modifications and describing what are the product variants.

FIG. 5 Schematic of the product variant determination approach

FIG. 6 is a list of physicochemical and in vitro biological characterization assays for comparability assessment and fingerprinting. Example is for trastuzumab biosimilar.

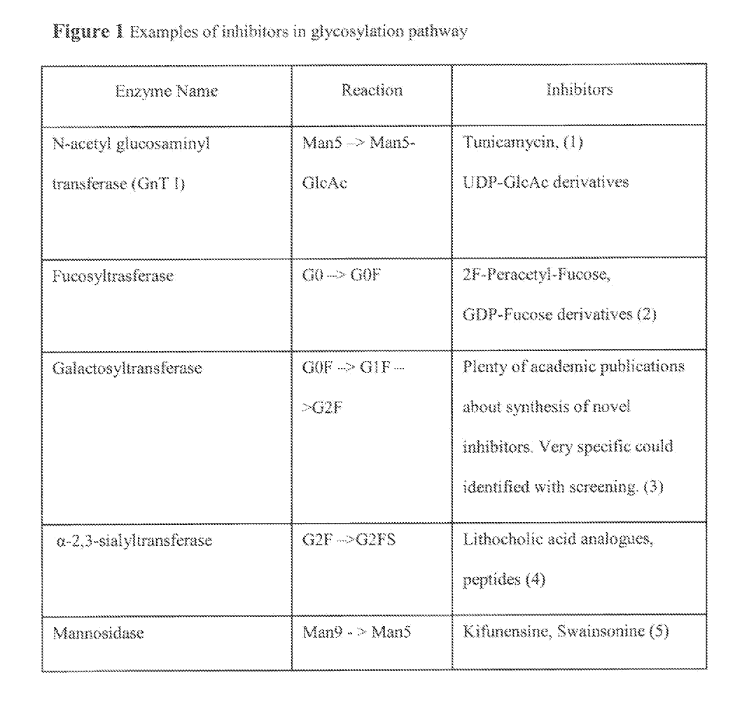

### DETAILED DESCRIPTION OF INVENTION

It is to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this invention pertains.

In describing and claiming the present invention, the following terminology and grammatical variants will be used in accordance with the definitions set forth below.

The term “fingerprinting,” is a method of analysis of a recombinant protein that results in full understanding of the product including but not limited to:
a. All product modifications
b. All product variants
c. Impact of product variants on biological activity (SAR equation)

The term “living cell,” as used herein, refers to cell used for production of a biosimilar version of a recombinant protein drug. Examples of a living cell include but are not limited to human, sheep, goat, cow, dog, cat, chicken, hamster, mouse, tobacco plant, and carrot sources. Examples of living cells which are commonly used to produce recombinant proteins as active drug ingredients include mammalian cells such as Chinese Hamster Ovary cells (CHO), murine myeloma NSO cells, Baby Hamster Kidney (BHK) cells. SP2/0, 293 or CAP-T cells.

The term “host proteins” refers to proteins present in living cells, which interact with and modify recombinant proteins expressed in said living cells.

The term “modulators” include small molecules, biological compounds, natural products, lipids that can modulate the activities of host proteins that can be added to the solution containing living cells that can specifically alter modifications on recombinant proteins. Modulators include both inhibitors and activators of host cell modification proteins. Modulator library refers to a collection of modulators that can be used to alter the activity of host proteins either to activate them or to inhibit them. The library of modulators may include small molecule drugs such as fucosyl transferase inhibitors, mannosidase inhibitor, biologic molecules such insulin, RNAi and RNA molecules, and other biomolecules known to those skilled in the art would recognize to affect post translational modifications of recombinant proteins or their biosimilars being produced in host cells.

In certain methods and embodiments one or more of the following compounds, known for purposes of this disclosure as Group I inhibitors, can be used to modulate modifications: 4,6,640 -trichloro-4,6,6′-trideoxy-1′,2-isopropylidene-3,3′,4′-tri-O -acetylgalactosucrose; hexa-O-benzoyl-4,6-O-isopropylidenesucrose; methyl 4,6-dichloro -4,6-dideoxy-α-D-galactopyranoside; methyl 2,3-di-O-tosyl-4,6-O-benzylidine-α-D -glucoside; 6′-chlorosucrose; 2,3,4-trichloro-2,3,4-trideoxy-1′,3′,4′,6′-tetraacetylgalactosucrose; 4,6-O-benzylidene-6′-acetylsucrose; myo inositol hexaacetate; 3,3′,4′6′-tetra-O-acetylsucrose; 3,4,6,3′,4′, 6′-hexa-O-acetylsucrose; 6,6′-diamino-6,6′-dideoxy-sucrose; D-glycero D-guloheptose; 2,3,1′,3′,4′6′-hexa-O-acetyl-4,6-O -orthobutyrylsucrose; 2,3,6,3′,4′-penta-O-acetyl-1′. 6′-di-O-tritylsucrose; 3,6,3′,6′-dianhydrotrehalose; 2,3,6,3′,4′-penta-O-acetyl-4-chloro-4-deoxy sucrose: 1,6-anhydro-3-nitro-3-deoxy-b-D-gulose; methyl 4,6-O-benzylidene sophroside; sucrose 4,6,1′,6′-tetratrityl 2,3,3′,4′-tetraacetate; 4,4′,6′,-trichloro-4,4′,6′-trideoxygalactosucrose; 4,6,1′,6′-tetrachloro-4,6,1′,6′-tetradeoxysucrose; trichlorogalactosucrose 6 teriary butyl diphenyl sialyl; 2,3:4,5-di-O-isopropylidine-β-D-fructopyranose; trichlorogalactosucrose 3′,4′lyxoepoxide triacetate; 6′ chloro-6′-deoxy-2,3,4,6,1′,3′,4′-hexa-O-acetylsucrose; 4,6,1′,6′-tetra-O-trytyl-2,3,3′,4′-O-acetylsucrose; 6,6′-dichloro-6-6′-dideoxysucrose; 3,4,6-trichloroglucose; isomaltulose octaacetate; 6-benozyl-1′,6′-ditosyl-2,3,4,3′,4′-penta-O -acetylsucrose; 2,3 dimethyl trichlorogalactosucrose triacetate; 1′,6′-dichloro-1′,6′-dideoxy -2,3,4,6,3′4′-hexa-O-acetylsucrose; 6,6′-di-O-triytyl-2,3,4,1′,3′,4′-hexaacetyl sucrose; octaacetyl α D-cellobiose; 6-chloro-6-deoxygalactose; 4,1′,4′,6′-tetrachloro-4,1′,4′,6′-tetradeoxy-2,3,6,3′-tetra-O-acetylgalactosucrose; 6-O-acetyl-1,2,-O-isopropylidine-α-D -glucofuranose; 2,3,4,6-tetra-O-trytyl glucose; 2,3:4,5-di-O-isopropylidinefructopyranosyl chloride; 4,6,6′-trichloro-4,6,6′-deoxy-3′,4′-anhydrosucrose; 6-chloro-6-deoxy -2,3,4,1′,3′,4′6′-hepta-O-acetylsucrose; N-octyl D-glucamine; 2,3,4,6-tetra-O-trytyl glucose; 1′,2:4,6-di-O-isopropylidine-3,3′6′-tri-O-acetyl sucrose; 2,3:4,6-di-O isopropylidine-3′,4′,6′-tri-O-benzoyl-1′-acetylsucrose; 1,2:4,6-di-O-isopropylidine-3,4′-di -O-acetyl-3′,6′-di-O-benzoylsucrose; 1′,2:4,6-di-O-isopropylidine-3,3′,4′,6′-tetra-O -acetylsucrose; 6-deoxy-6-carboxymethyl-1,2,3,4-tetra-O-trytyl glucospyranoside; 2,3,4,3′4′,6′-hexa-O-acetylsucrose; 1′,6′-dichloro-1′,6′-deoxy-2,3,4,6,3′,4′,6′-hexa-O -sucrose hexaacetate; 1′,2,4,6-di-O-isopropylidine sucrose; 3,4-anhydro-1,6-dichloro-1,6 dideoxy-β-D-lyxo-hexofuranosyl-3,6-anhydro-4-chloro-4-deoxy-α-D-galactopyranoside; 3,3′4′,6′-benzoyl sucrose; tetraacetyl glucuronic acid; 1,2,3,4,5-penta-O-acetylxylitol; benzyl β-D-fructopyranoside; 3,3′,4′,6′-tetra-O-cyclohexanoyl sucrose; phenyl β-D -galactoside; 2,3,4,6,1,2,3.6-octa-O-acetylmaltose; 2,3,4,6,1′,3′,4′-hepa-O-acetyl sucrose; 1′,2:4,6-di-O-isopropylidine-3,6′ diacetyl sucrose; β-D allose; 6′-chloro-6′-deoxy sucrose; 6-O-methyl-4,1′,6′trichloro4,1′,6′-trideoxygalactosucrose; 1′,4-di-O-mesyl-6′-O-benzoyl -2,3,6,3′,4′-penta-O-acetylsucrose; 6′-O-benzoyl-2,3,6,3′,4′-penta-O-acetylsucrose; 2,3,4,6,1′,3′,4′,6′-hexa-O-mesylsucrose; Methyl 4,6 O-benzylildene sophorose; Methyl 6-O-trytyl-2,3,4-tri-O-benzoyl-α-D-glucopyranoside; 6′t-butyldiphenylsilyl sucrose; 1,2:3,5-di-O-phenyl-6-deoxy-6-thioacetyl-α-D-glucofuranose; 1,3,4-tri-O-acetyl-6-chloro -2,6-dideoxy-α-D-glucoyranoside; 6-O-trytyl-1,2,3,4-tetra-O-acetyl-a-D-glucopyranoside; 4,6-O-isopropylidine-2,3,1′,3′,4′,6′-hexa-O-benzoyl sucrose; methyl 2,3-di-O-benzoyl-4,6-di-O-mesylglucopyranoside; 4,1′,6′-trichloro-4,1′,6′-trideoxy-2,3,6,3,4-penta-O-acetyl sucrose; methyl 4,6-O-benzylidine-2,3-di-O-tosyl-α-D-allopyranoside; 2,3,4,6-tetra-O -trytyl glucpyranose; methyl 4,6-O-benzylidine-2,3-di-O-tosyl-α-D-glucopyranoside; 1′,6′-Di-O-trytl-2,3,4,6,3′,4′-hexa-O-acetyl sucrose; 4,6:1′2-di-O-isopropylidine-3,3′,4′,6′-tetra-O-acetyl sucrose; 1′,2:4,6-di-O-isopropylidine sucrose; 6,3′,4′-tri-O-acetyl-4,1′,6′-trichloro-4,1′,6′-trideoxy galactosucrose; 6′-chloro-6′-deoxy sucrose; 7-O-trytyl 2,3,4,5,6-penta-O-acetyl-D-glycero-D-gulo-heptose diethyl dithio acetal; 6′-chloro-2,3,4,6,1′,3′,4′-hepta-O-acetyl sucrose; 3-acetamido-1,6-anhydro-2,4-d-O-acetyl-3-deoxy β-D-gulose; Methyl 3-benzymido-4,6-O-benzylidine-3-deoxy-α-D-altropyranose; 4,1′,6′-trichloro -4,1′,6′-trideoxy galactosucrose (sucralose); Methyl 3-acetamido-2,4-di-O-acetyl-3,6-dideoxy-α-L-hexoside; methyl 2,3-di-O-benzyl-4,6-di-O-mesylglucopryanoside; D-ribo -3,4,5,6-tetra-O-acetyl-1-nitro-hex-1-ene; 2-O-methyl-D-glucose diethyl dithio acetal; Methyl 3-acetamido-2,4,6-tri-O-mesyl-α-D-mannoside; D arabo-3,4,5,6 tetra-O-acetyl-1-nitro-hex-1-ene; 1,1-diethyl sulphonyl-(2-O-tosyl-α-D-arabinopyranosyl) methane hydrate; Methyl glucoside laurate; Methyl 2,3-anhydro-2,3-4,6-O-benzylidine-β-D -talopyranoside; Methyl 2,3-anhydro-4,6-O-benzylidine-β-D-talopyranoside; 3-acetamido -2,4-di-O-acetyl-1,6-anhydro-3-deoxy-β-D-idopyranose; 1,1-diethylsulphonyl-(3,4-O -isopropylidene-2-O-tosyl-α-D-arabinopyranosyl) methane hydrate; 2,3,4,5-tetra-O -benzoyl galactose; D-manno-3,7-anhydro-4-methoxy-5,6-isopropylidine-2,2-diethyl sulphonyl heptane; 2-acetamido-1,2-dideoxy-1-nitro-D-manitol; 1,1-diethylsulphonyl-L -arabo-2,2,4,5-tetrahydroxyhexane; 1′,6′-dichloro-1′,6′-deoxysucrose; Methyl 3-acetamido-3-deoxy-2,4,6-tri-O-acetyl α-D-mannopyranoside; Methyl 3-benzamido-4,6, -O-benzylidine-3-deoxy-2-O-mesyl-α-D-altropyranoside; Methyl 2-O-tosyl-4,6-O -benzylidene-α-D-glucopyranoside; 3 amino-1,6-anhydro-3-deoxy-β-D-altropyranose hydrochloride; Methyl 3-N-acetyl 3,6-dideoxy-2,4 di-O-acetyl-α-L-mannoside; Methyl 4,6-diazido-α-D-galactopyranoside; 6,4′,1″,6″-tetrachloro-6,4′,1″,6″-tetradeoxy raffinose; 6,6′dichloro-6,6′-dideoxy-3,4,3′,4′-tetra-O-acetyl-sucrose; 1,1-diethylsulphanyl 1-(α-D -lyxopyranosyl)-methane; D-xylo-3,4,5,6-tetra-O-acetyl-1-nitro-hex-1-ene; 1,1-diethylsulphanyl-1-(2,3,4 tri-O-acetyl-α-D-lyxopyranosyl)-ethane; 2,3,4,6-tetra-O-acetyl galactopyranose; 1-deoxy-1-nitro-D-glycerol-D-galactoheptitol; Methyl 4,6-diazido-2-O -benzoyl-3-O-mesyl α-D-glucopyranoside; 2-O-isopropylidien-3-acetamido-3-deoxy-α-D -allofuranose; 3,6-dideoxy-3-dimethylamine-L-mannose hydrochloride; 3-acetamido-1,2,4-tri-O-acetyl-3,6-dideoxy-β-L-glucopyranose; 2 (NHPO(OPh)2)-3,4,6 triacetyl glucosazide; 2,3,6,3′-tetraacetyl 4,1′,4′,6′ tetrachloro 4,1′,4′,6′ tetradeoxy galactosucrose; Arabinose diethyl mercaptal; 2-chloro-3-benzamino methyl hexaside; 1′-O-trytyl -2,3,4,6,3′,4′,6′-hepta-O-acetylsucrose; 2,1′-O-diphenyl silane3,4,6,3′,6′-hexa-O-acetyl sucrose; 2,3,4-trichloro-2,3,4-trideoxy fructose; D-glycero-D-guloheptose diethyl dithio acetal; 1 L-2-O-methyl-1-chiro-inositol pentabenzoate; Stevia glycoside; 4,1′,6′-trichlorotrideoxygalactosucrose tetraacetate OH-6; sucrose ethyl 4,6-orthoacetate hexaacetate; sucrose methyl 4,6-orthobutyrate hexaacetate; sucrose methyl 4,6-orthoacetate hexaacetate; 4,1′,6′-tribromotrideoxygalactosucrose pentaacetate; 6-0-benzoyl-4,1′,6′-trichlorotrideoxygalactosucrose tetraacetate; methyl 6-chloro-6-deoxy-α-D-galactopyranoside; methyl 4,6-dichloro-4,6-dideoxy-α-D-galactopyranoside; methyl 4,6-dichloro-4,6-dideoxy-α-D-glucopyranoside; 3,6:1′,4′:3′,6′-trianhydro-4-chloro-4-deoxygalactosucrose: 3′,6′-anhydro-4,6,1′-trichloro-4,6,1′-trideoxygalactosucrose; 4,1′,640 -trichlorogalactosucrose-3′,4′-lyxoepoxide triacetate; 4,6′-dichloro-4,6′-dideoxygalactosucrose hexaacetate; 4,1′,4′,6′-tetrachlorotetradeoxygalactosucrose tetraacetate; 6,1′,6′-trichlorotrideoxysucrose pentaacetate; 1′,6′-dichloro-1′,6′-dideoxysucrose pentaacetate OH-4; 4,6,1′,4′,6′-pentachloropentadeoxygalactosucrose triacetate; 4,6,1′,4′,6′-pentachloropentadeoxygalactosorbosucrose triacetate; 4,6,1′,4′,6′-pentachloropentadeoxygalactosucrose; 4,6,1′,4′,6′-pentachloropentadeoxygalactosorbosucrose; 6-0-acetyl-4,1′,6′-tribromo-4,1′,6-trideoxygalactosucrose; 1′,4′:3′,640 -dianhydro-4-bromo-4-deoxygalactosucrose; 4-bromo-4-deoxy-D-galactose; 3,6-di-0-benzoyl-1,2-0-isopropylidene-α-D-glucofuranoside; 3,6-di-0-benzoyl-1,2-0-isopropylidene-5-0-methyl-α-D-glucofuranos; 6-chloro-6-deoxy-1,2,-0isopropylidene-5-0-methyl-α-D-glucofuranos; trans-1,2-0-benzylidene-D-glycerol; cis -1,2-0-benzylidene-D-glycerol; cis-1,3-0-benzylidene-2-chloro-2-deoxy-D-glycerol; 4-0-mesyl-1′,6′-di-0-tritylsucrose pentaacetate; 6-chloro-6-deoxy-D-mannonolactone; 6-chloro-6-deoxy-D-mannonolactone triacetate; methyl 2-acetamido-2-deoxy-β-D -glucopyranoside; methyl 2-acetamido-2-deoxy-β-D-glucopyranoside triacetate; me 2-acetamido-6-chloro-2,6-dideoxy-β-D-glucopyranoside diacetate; 4-0-mesylsucrose pentaacetate OH-1′,6′; me 2-acetamido-6-chloro-2,6-dideoxy-α-D-glucopyranoside diacetate; 4-0-mesylsucrose heptaacetate; 3-0-acetyl-1,2:5,6-di-0-isopropylidene-α-D -glucofuranose; 3-0-acetyl-1,2-0-isopropylidene-α-D-glucofuranose; 3-0-acetyl-6-0-benzoyl-5-bromo-1,2-0-isopropylidene-β-L-idose; 3-0-acetyl-6-0-benzoyl-5-chloro-1,2-0-isopropylidene-α-D-glucose; 6-0-benzoyl-5-chloro-1,2-0-isopropylidene-α-D -glucofuranose; methyl 2-acetamido-6-chloro-2,6-dideoxy-α-D-glucopyranoside; 2-0-benzoyl-3-chloro-D-glyceraldehyde 2,4-dinitrophenylhydrazone; methyl 4,6-0-benzylidene-2-chloro-2-deoxy-α-D-mannopyranoside; methyl 3-0-benzoyl-4,6-0-benzylidene-α-D-glucopyranoside; methyl 3-0-benzylidene-2-chloro-α-D -mannopyranoside; 2-chloro-2-deoxy-D-mannitol; 4-(tetra-0-acetyl-β-D -glucopyranosyloxy)benzaldehyde; 6′-chloro-6′-deoxy-2,1′:4,6-di-0-isopropylidenesucrose; methyl 4,6-0-(p-nitrobenzylidene)-α-D-glucopyranoside diacetate; 4,6-0-(p -nitrobenzylidene)-α-D-glucopyranose triacetate; methyl 4,6-0-benzylidene-α-D -glucopyranoside diacetate; me 4,6-0-(m-nitrobenzylidene)-α-D-glucopyranoside diacetate (ax); 6,6′-dibromo-6,6′-dideoxysucrose hexaacetate; methyl 4,6-0-(m-nitrobenzylidene)-α-D-glucopyranoside (eq); 6,6′-diazido-6,6′-dideoxysucrose; me 4,6-0-(m -nitrobenzylidene)-α-D-glucopyranoside diacetate (eq); 6′-bromo-6′deoxysucrose heptaacetate, 6,8′-diamino-6,6′dideoxysucrose; methyl 6-0-(m-nitrobenzyl)-α-D -glucopyranoside; 6′amino-6′-deoxysucrose; 6-chloro-6-deoxy-D-glucitol pentaacetate; 1,2-0-isopropylidene-6-0-acetyl-α-D glucofuranose; 3,5-0-benzylidene-1,2-0-isopropylidene-6-O-acetyl-α-D-glucofuranose; methyl 3-0-benzoyl-4,6-0-benzylidene-2-chloro-α-D-glucopyranoside; 6-0-trityl-β-D-glucopyranose tetraacetate, 1,2,3,4-tetra-0-acetyl-β-D-glucopyranose; 6-deoxy-6-fluoro-β-D-glucopyranose tetraacetate; 3,5-benzylidene-1,2-O-isopropylidene-a-D-glucofuranose; 6-deoxy-6-fluoro-D-glucitol -pentaacetate; methyl 2,3,4,4-tri-0benzoyl-α-D-glucopyranoside; methyl 6-0-tosyl-α-D -glucopyranoside; methyl 2,3,4-tri-0-acetyl-6-thio-6-S-acetyl-α-D-glucopyranoside; 6-chloro-6-deoxy-D-glucitol (sy); 1,2,3,4-tetrα-0-acetyl-6-S-acetyl-6-thio-α-D -glucopyranose; 1,2,3,4-tetrα-0-acetyl-6-thio-α-D-glucopyranose dimer; 6-chloro-6-deoxy D-galactitol; 6-chloro-6-deoxy-D-galactitol pentaacetate; 1,2,5,6-tetra-0-benzoyl-3,4-0-isopropylidene-D-mannitol; 3,4-0-isopropylidene-D-mannitol; 1,2-0-isopropylidene-6-0-tosyl-α-D-glucofuranose (crude); 2,5-di-0-benzoyl-1,6-dichloro-3,4-0-isopropylidene-D -mannitol; 2,5-di-0-benzoyl-1,6-dichloro-D-mannitol; 1,2;3,5-di-0-benzylidene-6-0-tosyl -α-D-glucofuranose; 1,2;3,5-di-0-benzylidene-6-S-acetyl-α-D-glucofuranose; methyl 2,3-anhydro-4,6-benzylidene-α-D-gulopyranoside; 1,3:2,4:5,6-tri-0-ethylidene-D-glucitol; 1,3:2,4-di-0-ethylidene-D-glucitol; 5,6-anhydro-1,3:2,4-di-0-ethylidene-D-glucitol; 1,2:5,6-di-0isopropylidene-α-D-glucofuranose; 1,2:5,6-di-0-isoproylidene-α-D -allofuranose; 1,2-0-isopropylidene-α-D-allofuranose; 6-chloro-6-deoxy-1,2-0-isopropylidene-α-D-allofuranose; 6-chloro-6-deoxy-D-allose; 2,1′;4,6-di-0-isopropylidene sucrose tetraacetate; 1,2:5,6-di-0-isopropylidene-α-D-gulofuranose; 1,2-0-isopropylidene -α-D-glucofuranose; 1,2-0-cyclohexylidene-myo-inositol; 1,2-0-cyclohexylidene-myo -inositol tetraacetate; 6-chloro-6-deoxy-1,2-0-isopropylidene-α-D-glucofuranose; 3,4,5,6-tetra-0-acetyl-myo-inositol; 3,4,5,6-tetra-0-acetyl-myo-inositol hydrate; 3,4,5,6-tetra-0-acetyl-1-chloro-1-deoxy-scyllo-inositol; myo-inositol hexaacetate; 1-chloro-1-deoxy -scyllo-inositol pentaacetate; 1,2-dichloro-1,2-dideoxy-myo-inositol tetraacetate; 1-chloro -1-deoxy-scyllo-inositol; 3-0-benzoyl-1,2-5,6-0-di-isopropylidene-α-D-glucofuranose; methyl 6-chloro-6-deoxy-α-D-mannopyranoside triacetate; 3-0-benzoyl-1,2-0-isopropylidene-5,6-di-0-mesyl-α-D-glucose; methyl 4,6-0-benzylidene-α-D -mannopyranoside; methyl 2,3:4,6-di-0-benzylidene-α-D-mannopyranoside; 6-chloro-6-deoxy-D-mannose; methyl 4,6-0-benzylidene-2-chloro-2-deoxy-α-D-gIucopyranoside; 3,6-di-0-benzoyl-1,2-0-isopropylidene-5-0-mesyl-α-D-glucofuranose; 6-0-benzoyl-1-chloro-hexan-2,6-diol (syrup): 3,5,6-tri-0-benzoyl-1,2-0-isopropylidene-β-L-idofuranose; 6,6′-dichloro-6,6′-dideoxy-D-maltose hexaacetate; 3-0-acetyl-6-0-benzoyl-1,2-0-isopropylidene-5-0-mesyl-α-D-glucose; 3-0-acetyl-5,6-di-0-benzoyl-1,2-0-isopropylidene -β-L-idofuranose; 5,6-di-0-benzoyl-1,2-0-isopropylidene-β-L-idofuranose; phenyl 6-chloro-6-deoxy-β-D-glucopyranoside; 6′-chloro-6′-deoxysucrose pentaacetate OH-4,1′; 1,2-0-ethyleneβ-D-fructopyranoside; 6′-chloro-6′-deoxysucrose; methyl 6-chloro-6-deoxy-α-D-glucopyranoside triacetate; methyl 2,3-anhydro-4,6-0-benzylidene-α-D -allopyranoside; methyl 4,6-0-benzylidene-2,3-di-0-tosyl-α-D-glucopyranoside; methyl 4,6-0-benzylidene-α-D-altropyranoside; L-1,3,4,5,6-penta-0-benzoyl-2-0-methyl-chiro -inositol; 6-chloro-6-deoxy-α-D-altropyranose tetraacetate; 3,6-anhydro-1,2-0-isopropylidene-β-L-idofuranose 5-chlorosulphate; 3,6-anhydro-1,2-0-isopropylidene-β-L -idofuranose; 2-deoxyglucose; methyl 4,6-0-benzylidene-α-D-galactopyranoside; 4-chloro -4-deoxy-D-galactitol; methyl 4,6-0-benzylidene-2,3-di-0-tosyl-α-D-galactopyranoside; methyl 4,6-0-benzylidene-α-D-idopyranoside; 1,2-dichloro-1,2-dideoxy-myo-inositol; Benzyl 2-acetamido-4-0-(2-acetamido-2-deoxy-3,4,6-tri-0-acetyl-β-D-glucopyranosyl)-2-deoxy-3,6-di-0-acetyl-β-D-glucopyranoside; 4′-chloro-4′deoxysucrose hexaacetate OH-3′; 6-chloro-6-deoxy-1,2-0-isopropylidene-β-D-fructofuranose; 6,6′-dichloro-6,6′-dideoxysucrose pentaacetate OH-1′; 2-chloroethyl β-D-fructopyranoside; 6-chloro-2,6-dideoxy-α-D-glucopyranose triacetate; 4,6-0-benzylidenesucrose hexaacetate; 5,6-dichloro-5,6-dideoxy-1,2-0-isopropylidene-β-L-talofuranose; 5,6-dichloro-5,6-dideoxy-β-L-talofuranose; Methyl neuraminic acid-5-acetyl-chloride ethyl xanthate; Benzyl 2-acetamido-3,6-di-0-benzyl-2-deoxy-4-0-(3,4,6-tri-0-benzyl-β-D-mannopyranosyl)-α-D -glucopyranoside; Benzyl 4-0-β-D-galactopyranosyl-β-D-glucopyranoside heptaacetate; Benzyl 2-acetamido-4-0-(2-acetamido-2-deoxy-β-D-glucopyranosyl)-2-deoxy-β-D -glucopyranoside; Benzyl 2-acetamido-3,6-di-0-benzyl-2-deoxy-4-0-(3,4,6-tri-0-benzyl-β-D-arabinohexopyran-2-ulosyl)-α-D-glucopyranoside; Ethyl-4,6-0-benzylidene-2-deoxy-2-phthlamido-1-thio-β-D-glucopyranoside; 4,6:2,1′-di-0-isopropylidenesucrose tetraacetate; 3,3′,4′,6′-tetra-0-acetylsucrose; 3′,4′-di-0-acetyl-4,1′,6′-trichlorotrideoxygalactosucrose; methyl 4-chloro-4-deoxy-α-D-galactopyranoside; 3,1′,4′,6′-tetrachloro-3,1′,4′,6′-tetradeoxyallosorbosucrose; methyl 6-chloro-6-deoxy-α-D-glucopyranoside; galactosucrose; 1′,6′-dichloro-1′,6′-dideoxysucrose hexaacetate; 6,6′-dichloro-6,6′-dideoxysucrose tetraacetate OH-2,1′; 2,3-0-isopropylidene-6,1′,6′-tri-0-tritylsucrose triacetate; 3-0-acetyl-3′,6′-di-0-benzoyl-4,6:2,1′-di-0-isopropylidenesucrose; 4,6:2,1′-di-0-isopropylidenesucrose tetrabenzoate; 4,1′,6′-tri-0-mesylsucrose pentaacetate; 4-0-mesylsucrose heptaacetate; 3-acetamido-5,6-di-O-acetyl-1,2-isopropylidene-α-D -allofuranose; methyl 2-acetamido-3-O-acetyl-4,6-di-O-mesyl-α-D-glucopyranoside; -methyl 4,6-O-benzylidene-2,3-imino-α-D-mannopyranoside; methyl 4,6-O-benzylidene -2,3-imino-N-p-nitrobenzoyl-α-D-alloside; methyl 3-acetamido-4,6-O-benzylidene-2-O -mesyl-α-D-altropyranosid; methyl 2,3-anhydro-4,6-O-benzylidene-β-D-talopyranoside; methyl N-acetyl-4,6-O-benzylidene-2,3-imino-α-D-mannopyanoside; methyl 4,6-O -benzylidene-α-D-sophoroside tetraacetate OH-3; methyl 2-O-benzoyl-4,6-O-benzylidene -α-D-glucopyranoside; Ethyl-3-0benzyl-2-deoxy-2-phthlamido-1-thio-β-D -glucopyranoside; methyl 6,6′-dichloro-6,6′-dideoxy-β-D-cellobioside; methyl 2,3-di-O -acetyl-4-O-mesyl-6-thiocyanato-α-D-galactoside; methyl 3-acetamido-3-deoxy-2,4,6-tri -O-mesyl-β-D-glucopyranoside; Me N-acetyl-4-6-O-benzylidene-2,3-dideoxy-2,3-imino-α-D-alloside; Me 4,6-O-benzylidene-2,3-imino-N-(2,4-dinitrophenyl)-α-D-alloside; lactose octaacetate (α/β); Chitobiose oxazoline hexaacetate; hexadecyl 3′,4′-0-isopropylidene-β-D-lactoside; methyl 4,6-0-isopropylidene-β-D-glucopyranoside; hexadecyl β-D-lactoside; tetracosyl β-D-lactoside; methyl 3-deoxy-3-fluoro-4,6-0-isopropylidene-β-D -allopyranoside; methyl 3-deoxy-3-fluoro-β-D-allopyranoside; 2-deoxy-2-fluoro-1,3,5-tri -0-(4-chlorobenzoyl)-α-D-ribofuranose; p-Mephenyl 2-azido-346-tri-0-p-chlorobenzyl-1-thio-β-D-galactosid; hexadecyl β-D-lactoside pentaacetate OH-3′,4′; methyl 2,3,6-tri-0-benzoyl-α-D-galactopyranoside; Allyl-β-D-chitobioside; trichloroethyl 2-acetamido-2-deoxy-α-D-glucopyranoside triacetate; trichloroethyl 2-acetamido-2-deoxy-β-D -glucopyranoside triacetate; tce 2-acetamido-3-benzoyl-4,6-orthoacetyl-β-D -glucopyranoside; trichloroethyl β-D-chitobioside heptaacetate; (2′,2′,2′-trichloroethyl) 2-acetamido-2-deoxy-3-0-benzoyl-6-0-acetyl-β-D-glucopyranoside; allyl β-D-chitobioside heptaacetate; 3,4,6-tri-O-benzyl-D-mannose; tetrα-O-benzoyl α-D-glucopyranosyl bromide; tetra-O-benzoyl-2-hydroxy-D-glucal; 3,4,6-tri-O-benxoyl-α-D-hexopyranos-2ulosyl bromide; benzyl α-D-manno(1α3)bioside 6-chloroacetate hexabenzoate; benzyl α-D-manno(1α3)bioside 6-OH hexabenzoate; 2-deoxy-2-phthalimido-β-D-glucosamine tetraacetate: 4-deoxy-4-fluoro-D-galactose: benzyl 2-acetamido-2-deoxy-α-D -glucopyranoside; benzyl 2-acetamido-4;6-O-benzylidene-2-deoxy-α-D-glucoside; benzyl α-D-mannopyranoside; Ethyl-6-0-acetyl-3-0-benzyl-2-deoxy-2-phthlamido-1-thio-β-D -glucopyranoside; benzyl 2-acetamido-6-O-acetyl-3-O-benzoyl-2-deoxy-α-D-glucoside; benzyl 2-acetamido-3-O-benzyl-4,6-O-benzylidene-α-D-glucoside: EtS 2-O-(2-acetamido-β-D-glucopyranosyl)-α-D-mannoside hexaacetate; Benzyl 2,4-di-benzoyl-a-D -mangopentaoside tetradecaacetate; Benzyl 2,4-di-0-benzoyl-3-0-[2-0-(2-acetamido-2-deoxy-3,4,6-tri-0-acetyl-β-D-glucopyranosyl)-3,4,6-tri-0-acetyl-α-D-mannopyranosyl]-α-D-mannopyranoside; Benzyl 2-acetamido-3-0-(tetra-0-acetyl-β-D-galactopyranosyl)-4,6-0-benzylidene-2-deoxy-α-D-glucopyranoside; Benzyl 2-acetamido-3-0-(tetra-0-acetyl-β-D-galactopyranosyl)-2-deoxy-α-D-glucoside; 1,2:5,6-di-0-isopropylidene-α-D -galactofuranose; 2-O-acetyl-3,4,6-tri-O-benzyl-β-D-glucopyranose; Benzyl 2-acetamido-4-0-(2-0-acetyl-3,4,6-tri-0-benzyl-β-D-glucopyranosyl)-3,6-di-0-benzyl-2-deoxy-α-D -glucopyranoside; benzyl 2-acetamido-3,6-di-O-benzyl-2-deoxy-α-D-glucopyranoside; Benzyl 2-acetamido-3,6-di-O-benzyl-4-O-(3,4,6-tri-O-benzyl-β-D-glucopyranosyl)-2-deoxy-α-D-glucopyranoside; 2-O-β-D-glucopyranosyl-D-glucopyranose; Benzyl 4-0(3,4-0-isopropylidene-β-D-galactopyranosyl)-β-D-glycopyranoside; 2-0-α-D-mannopyranosyl -3,4,6-tri-0-benzyl-D-mannopyranose; 4-methylphenyl 1-thio-β-D-lactoside heptaacetate; 4-methylphenyl 4-0-(2,6di-0-acetyl-β-D-galactopyranosyl)-2,3,6-tri-0-acetyl-1-thio-β-D -glucopyranoside; 4-methylphenyl 4-0-(3,4-0-isopropylidene-β-D-galactopyranosyl)-1-thio-β-D-glucopyranoside; Ethyl 3-0-benzyl-2-deoxy-2-phthalimido-4-0-β-D -galactopyranosyl-1-thio-β-D-glucoside; Ethyl 2-acetamido-6-0-acetyl-3-0-allyl-2-deoxy -4-0-(tetra-0-acetyl-β-D-galactopyranosyl) 1-thio-β-D-glucopyranoside; Benzyl 2-acetamido-6-0-acetyl-3-0-benzyl-2-deoxy-α-D-glucopyranoside; Benzyl 2,4-di-0-benzoyl -6-0-(tetra-0-benzoyl-α-D-mannopyranosyl)-α-D-mannopyranoside; Benzyl 2-acetamido -6-0-acetyl-2-deoxy-3-0-(tetra-0-acetyl-β-D-galactopyranosyl)-α-D-glucopyranoside; Benzyl 2-acetamido-6-0-acetyl-3-0(tetra-0-0-acetyl-β-D-galactopyranosyl)-4-0-(tri-0-benzyl-α-L-fucopyranosyl)-2-deoxy-α-D-glucopyranoside; 1,4,6-tri-0-acetyl-3-0-(tetra-0-acetyl-α-D-galactopyranosyl)-α-D-galactopyranose; 1,4,6-tri-0-acetyl-2-0-(tri-0-benzyl-α-L-fucopyranosyl)-3-(0-(tetra-0-acetyl-α-D-galactopypyranosyl)-α-D-galactopyranose; Benzyl 4,6-0-benzylidene-α-D-glucopyranoside; Benzyl 2,3-di-0-benzyl-4,6-0-benzylidene-α-D -glucopyranoside; Benzyl 2,3-di-0-benzyl-α-D-glucopyranoside; Benzyl 0-α-D -galactopyranosyl-(1→3)-0-β-D-galactopyranosyl-(1→4)2,3-di-0-benzyl-α-D -glucopyranoside; Benzyl 2-acetamido-3-0-benzyl-2,6-dideoxy-6-iodo-α-D -glucopyranoside; Benzyl 2-acetamido-3-0-benzyl-2,6-dideoxy-α-D-glucopyranoside; Benzyl 2-acetamido-6-0-acetyl-3-0-benzyl-2-deoxy-α-D-glucopyranoside; Phenyl 2,3,4,6-tetra-0-acetyl-1-thio-α-D-mannopyranoside; 1,3,4,6-tetra-0-acetyl-β-D-mannopyranose: 1,2,3,6-tetra-0-benzoyl-4-0-(2,3-di-0-benzoyl-4,6-0-isopropylidene-β-D -galactopyranosyl)-α& β-D-glucopyranose; 1,2,3,6-tetra-0-benzoyl-4-0-(2,3-di-0-benzoyl -β-D-galactopyranosyl)-β-D-glucopyranose; 1,2,3,6-tetra-0-benzoyl-(2,3,6-tri-0-benzoyl-β-D-galactopyranosyl)-β-D-glucopyranose; 1,2,3,6tetra-0-benzoyl-4-0-(2,3-di-0-benzoyl-β-D-galactopyranosyl)-α-D-glucopyranose; 2,3,6-tetra-0-benzoyl-(2,3,6-tri-0-benzoyl-β-D-galactopyranosyl)-α-D-glucopyranose; Phenyl 2,3,6-tri-0-benzoyl-1-thio-β-D -galactopyranoside; Phenyl 3,6-di-0-benzoyl-1-thio-β-D-galactopyranoside; Phenyl 1-thio -β-D-galactopyranoside; Benzyl 4-0-(4,6-0-4-methoxybenzylidene-β-D-galactopyranosyl) -β-D-glucopyranoside; Benzyl 4-0-2,3-di-0-acetyl-4,6-0-4-methoxybenzylidene-β-D -galactopyranosyl)-2,3,6-tri-0-acetyl-β-D-glucopyranoside; Benzyl 4-0-(2-0-acetyl-3,4-0-isopropylidene-6-0-4-methoxybenzyl-β-D-galactopyranosyl)-2,3,6-tri-0-acetyl-β-D -glucopyranoside; Benzyl 4-0-(2-acetyl-β-D-galactopyranosyl)-2,3,6-tri-0-acetylβ-D -glucopyranoside; 2,3,6,3′,4′-penta-0-acetylsucrose; (4-methyl phenyl)sulphenyl 2-azido -3,4,6-tri-0-(4-chlorobenzyl)-2-deoxy-β-D-galactopyranoside; 4,6-0-)4-methoxybenzylidene)-2-acetamido-2-deoxygalactopyranose; Benzyl 2-acetamido-2-deoxy-3,6-di-0-benzyl-α-D-glucopyranoside; Benzyl 4-0-(4,6-0-benzylidene-β-D -galactopyranosyl)-β-D-glucopyranoside; Benzyl 2,3,6-tri-0-benzyl-4-0-(2,3-di-0-benzyl -4,6-0-benzylidene-β-D-galactopyranosyl)-β-D-glucopyranoside; Benzyl 2,3,6-tri-0-benzyl-4-0-(2,3,6-tri-0-benzyl-β-D-galactopyranosyl)-β-D-glucopyranoside; Ethyl 4,6-0-benzylidene-2-deoxy-2-phthalimido-1-thio-β-D-galactopyranoside; Benzyl 2,3-di-0-benzyl-4,6-0-benzylidene-β-D-galactopyranoside; Benzyl 2,3-di-0-benzyl-4,6-0-benzylidene-β-D-galactopyranoside; 3-0-(2-acetamido-2-deoxy-α-D-galactopyranosyl)-D -galactose; 3-0-(2-acetamido-2-deoxy-α-D-galactopyranosyl)-D-galactose; 1,3,4,6-tetra-0-acetyl-2-deoxy-2-phthalimido-D-glucopyranose; Methyl 3,4,6-tri-0-acetyl-2-deoxy-2-phthalimido-β-D-galactopyranoside; Methyl 4,6-0-benzylidene-2-deoxy-2-phthalimido-3-O-(3,4,6-tri-O-acetate-a-galactopyranoside-1,2-orthoacetyl)-β-D-galactopyranoside; Methyl 4,6-0-benzylidene-2-deoxy-2-phthalimido-β-D-galactopyranoside; 1,2,4,6-tetra-0-acetyl-3-0-(2,3,4,6-tetra-0-acetyl -α-D-glucopyranosyl)-α-D-glucopyranose; Thiophenyl 2,3,4,6-tetra-0-benzyl-β- D-galactopyranoside; 2,3,4,6-tetra-0-benzyl-D-galactose; Methyl 2-chloro-3-acetamido-2,3-dideoxy-a-D-altropyranoside; Methyl 3-acetamido-2,3-dideoxy -4,6-isoprpylidene-a-D-glucopyranoside; Methyl 2,3-anhydrodideoxy-2,3-acetamido-4,6-O-benzylidene-a-D-allopyranoside; Methyl 2,3-dideoxy-3-acetamido-4,6-di-O-mesyl-a -D-glucopyranoside; Methyl 3-aminohydrochloride-3-deoxy-4,6-benzylidene-a-D -mannoside; 2,1′-isoprpylidene-2′,3′,4′-tri-O-acetyl sucrose; Methyl a-D-galactoside; Gamma-D-Galactonolactone.

The term “recipe” refers to a mixture of the modulators and their concentrations that will be used to produce said recombinant protein or biosimilar with the target profile.

The term “recombinant protein” refers to any protein species, produced in living cells, systems, or organisms resulting from recombinant DNA technology. As used herein, the term “recombinant protein” includes but it is not limited to, proteins, polypeptides, and monoclonal or polyclonal antibodies and their biosimilar versions.

As used herein the term “antibody” encompasses whole antibodies including single chain antibodies, and antigen whole antibodies, and antigen binding fragments thereof. Fab, Fab′ and F(ab′)2, Fd, single chain Fvs (scFv), single chain antibodies, disulfide-linked Fvs (sdFv) and fragments comprising either VL and VH are all within the present definition of the term “antibody.” Antibodies may originate from any animal origin including birds and mammals. Preferably, the antibodies are human, murine, rabbit, goat, guinea pig. camel, horse, or chicken.

The term “biosimilar” refers to a recombinant protein, commonly with identical amino acid sequence to a reference commercial product that contains, similar, very similar to or same post-translational modifications as the reference product yielding similar biological activity to that product.

The term “reference product” refers to a currently or previously marketed recombinant protein, also described as the “originator” or “branded product” serving as a comparator in the studies. An “originator” or “branded” product are examples of a reference product.

The term “reference standard” refers to a highly characterized drug substance. The reference standard is prepared during the drug development cycle to serve as a comparator to all subsequent lots being manufactured.

The term “biobetter” refers to a version to an original biological drug with the same protein sequence but post-translational modifications that are outside the target profile range, which affect the drug's biodistribution, pharmacokinetics and pharmacodynamics.

As used herein, the term “candidate” with reference to biosimilar drug or bio-better drug, refers to the intent that it will be the subject of an application for commercial sale submitted for approval by one or more drug regulatory agencies in one or more different jurisdictions.

Recombinant proteins generally contain post-translational modifications. These modifications include but are not limited to: glycosylation, carboxylation, hydroxylation, 0-sulfation, amidation, glycosylation, glycation, alkylation, acylation, acetylation. phosphorylation, biotinylation, formylation, lipidation, iodination, prenylation, oxidation, palmitoylation, pegylation, phosphatidylinositolation, phosphopantetheinylation, sialylation, and selenoylation.

The term “glycosylation” refers to attachment of oligosaccharides to proteins and represents the most commonly found post-translational modification of recombinant proteins. Oligosaccharides consist of monosaccharide units that are connected to each other via glycosidic bonds. Oligosaccharides may also be branched, with each of the sugar units in the saccharide serving as an optional branching point. The oligosaccharide chains are attached to proteins co-translationally or post-translationally, via specific asparagine (N-linked) or serine-threonine (0-linked) residues. For N-linked glycosylation the consensus amino acid sequence of recombinant protein is Asn-X-Ser/Thr. 0-sulfation entails the attachment of a sulphate group to tyrosine, serine and threonine residues mediated by sulfotransferases. Amidation is characterized by the replacement of the C-terminal carboxyl group of a protein with an amide group, y-carboxylation and -hydroxylation modifications are mediated by specific carboxylase and hydroxylase enzymes, with conversion of target glutamate residues toy- carboxyglutamate (Glu - - - + Gla) and either target conversion of aspartate residues to -hydroxyaspartate (Asp - - - + Hya) or asparagine residues to -hydroxyasparagine (Asn - - - + Hyn).

The phrase “modifications on the recombinant protein are substantially the same as the post-modifications the reference protein” can be taken to mean that the levels of post-translational modifications are within the ranges of the post-translation modifications identified in at least five lots of the reference protein.

The method for developing “target profile” and ‘target profile range” or “target range” as described in Examples 1 and 2.

The disclosed method involves developing a media recipe from growing cells to produce a recombinant protein of interest. The media can be any medium dial is appropriate for growth of the cells that are used to produce the recombinant protein.

The media can include supplements of which concentrations may be known or unknown. Examples of suitable supplements include salts, amino acids, vitamins, lipids, glutamine, glucose and galactose. Growth media for cells can be made custom or purchased commercially from companies like Gibco, Lonza, Millipore. Hyclone, GE and others familiar to those skilled the art of upstream process media development

Any cell that can be used for the production of the target recombinant protein can be used in the present method. Suitable cells generally will excrete the produced protein into the medium from which the recombinant protein can be isolated. Most commonly used cells are all variants of CHO cells. CAP-T cells, murine myeloma NSO cells. Baby Hamster Kidney (BHK) cells. SP2/0 cells, 293 cells or NSO cells.

The cells can be grown as a batch, as in shake flasks, or in any type and size of bioreactor and/or wave bags for production of the recombinant protein. Manufacturers of growth chambers and apparatuses include but are not limited to those produced by Millipore, General Electric, Eppendorf (New Brunswick), and Sartorius Stadium.

When cultured in a bioreactor. a control mechanism for altering conditions for production of the recombinant protein may be also provided. The mechanism for altering conditions may be in digital data communication with the controller so that an operator may alter production conditions by providing input to the controller. Conditions which may be altered using the controller include, but are not limited to: temperature, pressure, gas flow, agitation, and composition of growth medium components. Examples of growth medium components include, but are not limited to carbohydrates, salts, proteins and lipids and one or more components from the modulator library.

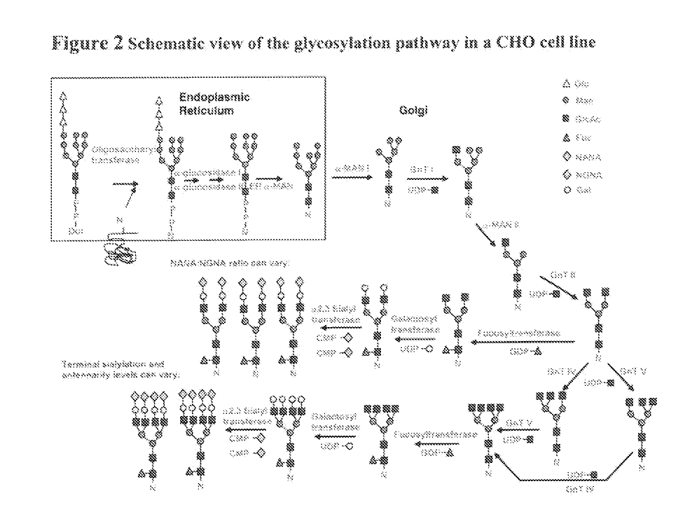

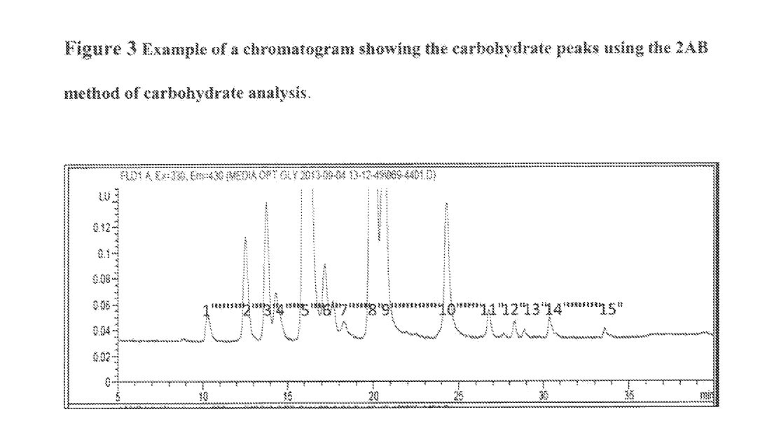

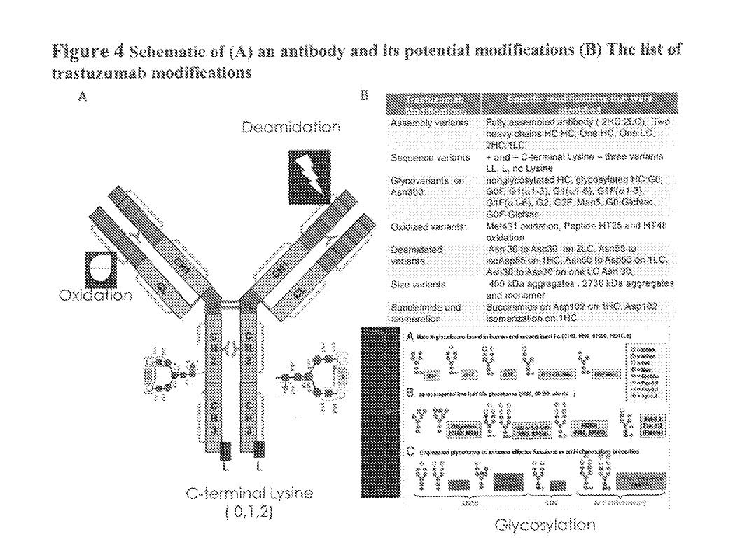

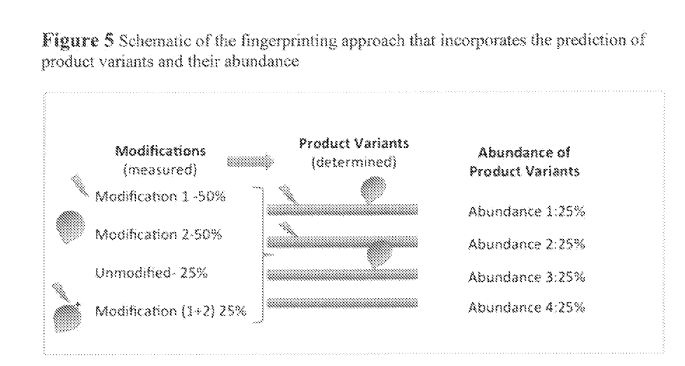

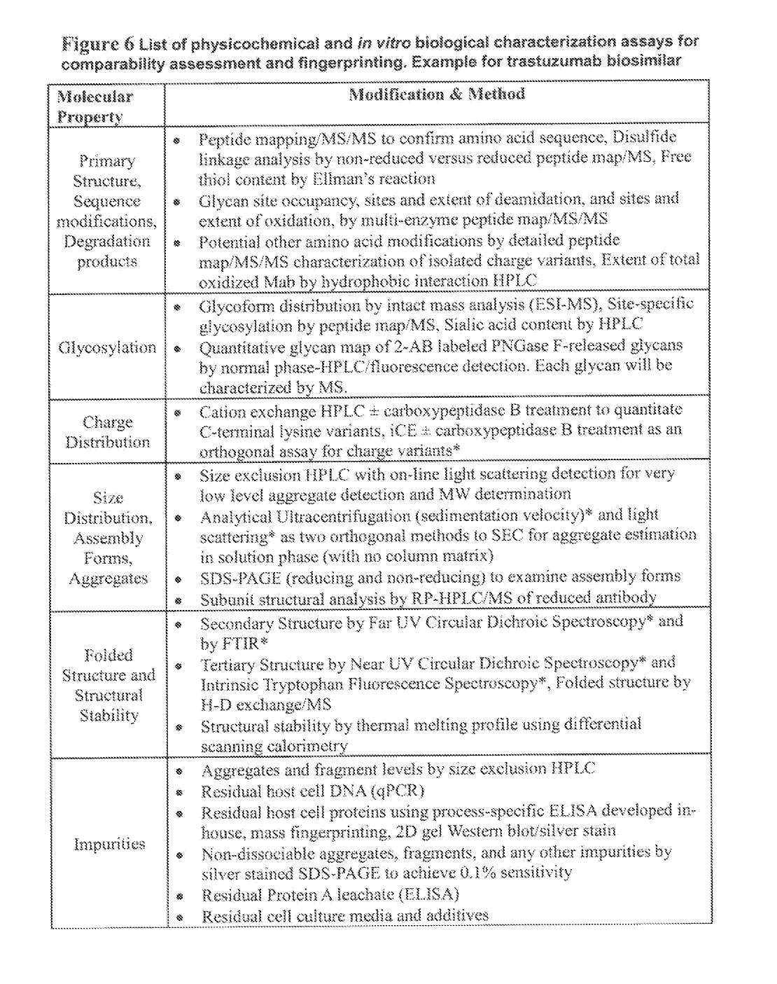

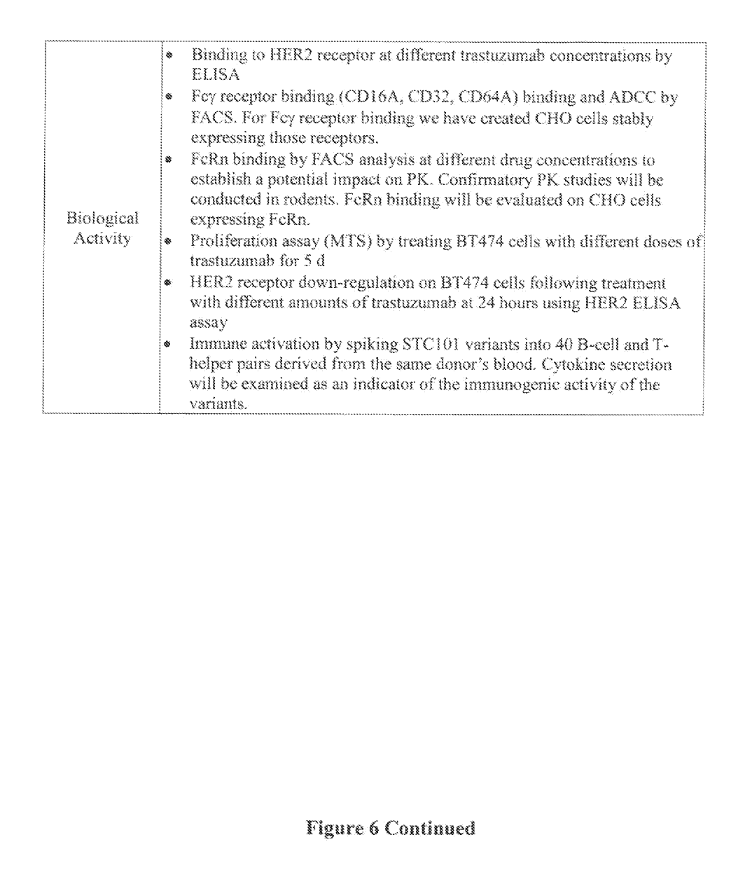

Any modification that can be controlled by the addition or removal of a modulator is amenable to modulation by the present methods. Glycosylation is an example of a modification that is particularly amendable to the optimization by the present methods as the host proteins involved in the glycosylation pathway are well known (FIG. 2) and can be modulated by a variety of inhibitors (FIG. 2). Other modifications are described in the definition section.

Any suitable method known to one skilled in the analytical arts can be used for measuring the levels of modifications. Mass spectrometry (MS) is a powerful method for analyzing and quantifying modifications. Some of the MS based methods amenable to said analysis may include but are not limited to: intact mass analysis, reduced mass analysts, peptide map analysis, and disulfide linkage analysis. Intact mass analysis by ESI-MS is used for identification and quantitation of modifications on a recombinant protein including but not limited to glycosylation and C-terminal lysine content. To analyze complex molecules such as antibodies, reduced mass analysis and peptide mass analysis should provide detailed information including the exact amino acid that has been modified. To conduct reduced muss analysis heavy and light chains of the antibody are first reduced, then resolved using reverse phase chromatography or other methods known to one skilled in the art and subsequently analyzed using ESI-MS. To conduct a peptide map analysis, an antibody is first digested with an enzyme that leads to antibody fragmentation. Each peptide is first resolved on appropriate chromatographic media and then analyzed by ESI-MS for amino acid sequence and modification such us glycosylation, deamidation, oxidation, disulfide scrambling, and C-terminallysine content. Enzymes that can be used for recombinant protein digestion include but are not limited to trypsin and Lys-C.

Chromatography by HPLC or UPLC is another powerful method to analyze recombinant proteins. For example, glycan species can he quantitated using a fluorescent 2AB labeling method. In this method, glycans are first removed from the protein by digestion with N-glycanase and then a fluorescent label is added to each glycan. The glycans can then be resolved using HILIC based chromatography and quantitated by measuring relative area under the curve. For oxidation quantitation an HIC based method can be used.

To determine the level of deamidation using chromatography based methods ISOQUANT Isoaspartate Detection Kit can be used. The ISOQUANT Isoaspartate Detection Kit uses the enzyme Protein Isoaspartyl Methyltransferase (PIMT) to specifically detect the presence of isoaspartic acid residues on a recombinant protein. PIMT catalyzes the transfer of a methyl group from S-adenosyl-L-methionine (SAM) to isoaspartic acid at the a-carboxyl position, generating S-adenosyl homocysteine (SAH) in the process. SAH formation is then quantitated in the sample by comparing it to the standard provided in the kit.

The present invention provides methods to identity, characterize, quantify, remove, and assemble product variants to produce a biosimilar that exhibits fingerprint level of similarity to the originator.

In one aspect of the invention, there is provided a method for producing a biosimilar product showing a fingerprint level similarity to the originator as follows:
(a) Establishing a relationship between product modifications and biological activity;
   i. Identifying the number (n) of modifications present on a recombinant protein;
   ii. Preparing a recombinant protein enriched for one or two modifications at the time at least at three different levels (high, medium, low) for a total of 3n enriched variants produced;
   iii. Confirming the identity of each enriched variant using HPLC and MS based assays;
   iv. Measuring biological activity of the enriched recombinant protein generated in ii). using biological assays relevant for said recombinant protein;
   v. Establishing a relationship between the modification and the biological activity;
(b) Measuring the quantity and type of specific modifications found on the at least three originator hatches using analytical assays;
(c) Setting target profile for the modifications of the originator based on data generated in b).
(d) Growing living cells expressing the biosimilar with the identical aminoaeid sequence to the originator;
(e) Isolating the biosimilar from d) and comparing its modifications to the target profile set in c).
(f) Selecting a plurality of growth media and one or more modulators to change modifications on the biosimilar and growing the cells in the presence of said modulators. Modulators can be selected from the library of modulators;
(g) Isolating the product from f). and comparing its modifications to the target profile in c).
(h) Repeating steps f), g) with additional modulators and or at different modulator concentrations to match modifications set in b). The modulators can be used alone or in a combination with each other. The set of exact imHlulution required to obtain the target profile provides a recipe for the production of said biosimilar. Target profile should not set be outside the specifications set for said originator;
(i) Once the cell culture production process is optimized, isolating the optimized product through a series of purifications steps which include but are not limited affinity, ion exchange or mixed mode chromatography with a goal to remove specific product variants:
(j) Measuring the quantity and type of specific modifications found on the biosimilar and comparing it to the target in b);
(k) Determining product variants for each product hatch using analytical data produced in b), and j);
(l) Comparing the type and quantity of the biosimilar product variants to the range of product variants produced by a originator;
(m) Determining the impact of each product variant on biological activity based on the structure activity relationship; summing up the biological activity of all variants based on their relative concentration to identify whether the biological activity of the biosimilar is within the range for the predicted biological activity the originator;
(n) If specific product variants need to be removed, selecting a plurality of growth media and one or more modulators to change modifications on the biosimilar and growing the cells in the presence of said modulators. Modulators can be selected from the library of modulators; Isolating the product from n). through a series of purifications steps which include but are not limited affinity, ion exchange or mixed mode chromatography with a goal to remove specific product variants;
(o) Confirming that biological activity of the biosimilar is within 80 to 125% of the originator in in vitro and in vivo biological assays;

In another aspect of the invention, there is provided a method for a process change for an originator with a fingerprint level similarity to the reference standard:
(a) Establishing a relationship between product modifications and biological activity;
   i. Identifying the number (n) of modifications present on a recombinant protein;
   ii. Preparing a recombinant protein enriched for one or two modifications at the time at least at three different levels (high, medium, low) for a total of 3n enriched variants produced
   iii. Confirming the identity of each enriched variant using HPLC and MS based assays;
   iv. Measuring biological activity for the recombinant protein variants generated in ii). using biological assays relevant for said recombinant protein;
   v. Establishing a relationship between the modification and the biological activity;
(b) Measuring the quantity and type of specific modifications found on the reference product or alternatively using product specifications to set the target range;
(c) Growing living cells expressing the originator in a presence of growth media that produces higher tiler or other beneficial cell line characteristics;
(d) Selecting a plurality of one or more modulators to change modifications on the originator produced using a new process and growing the cells in the presence of said modulators. Modulators can be selected from the library of modulators;
(e) Isolating the product from d). and comparing its modifications to the target set in b);
(f) Repeating steps d), e) with additional modulators and or at different modulator concentrations to match modifications set in b). The modulators can be used alone or in a combination with each other. The set of exact modulators and concentrations required to obtain the target profile provides a recipe for the production of said comparable biologic. The target profile should not be set outside the specifications set for said originator;
(g) Once the cell culture production process is optimized, isolating the optimized product through a series of purifications steps which include but are not limited affinity, ion exchange or mixed mode chromatography with a goal to remove specific product variants;
(h) Measuring the quantity and type of specific modifications found on the originator produced using a new production process and comparing it to the target in b);
(i) Determining product variants for each product batch using analytical data produced in b). for the reference product and in h). for the originator produced using a new production process.
(j) Comparing the type and quantity of the originator product variants produced using new optimized process to the range of product variants produced by the original process;
(k) Determining the impact of each product variant on biological activity based on the structure activity relationship; adding the biological activity of all variants based on their relative concentration to identify whether the theoretical biological activity of the originator produced using a new process is within the range for the original process;
(l) If specific product variants need to be removed, selecting a plurality of growth media and one or more modulators to change modifications on the originator produced using the new process and growing the cells in the presence of said modulators. Modulators can be selected from the library of modulators; Isolating the product from n). isolating the Optimized product through a series of purifications steps which include but are not limited affinity, ion exchange or mixed mode chromatography with a goal to remove specific product variants;
(m) Confirming that biological activity of the originator produced using new process is within 80 to 125% of the originator produced using the original process;

The described method results in the development of a recipe for media having concentrations of a variety of modulators that are required to produce recombinant proteins matching a target profile. The recipe is ideally used to produce the recombinant protein after a manufacturing process change or during biosimilar development. The method is particularly useful in the development of biosimilar products having modifications that are difficult to match and have the advantage that they can be used while keeping cell productivity high because the method decouples the productivity from target profile. Examples where the method can be used include trastuzumab biosimilar.

### EXAMPLE 1

### Setting A Target Profile

This example demonstrates one method for identifying a target profile for development of a recipe for production of a recombinant protein. In order to identify target profile or target profile range, at least 3-5 batches of the original reference product should be examined for the type and the amount of specific modifications. For biosimilar development a reference is defined as reference product. For a process change, a reference is defined as one batch of the reference standard and an additional 4 batches of the product made using the original process. In the example below to set target modifications for biosimilar development. 5 hutches of the reference product were analyzed for modifications. Out of 14 modifications, two modifications (glycosylation—G0 and glycosylation G2 were not observed. Other modifications were measured and are shown in Table 1 to be present at different levels on different batches. To set the target profile, first the exact measurements for each modification are identified for all five batches 1-5. For example, for Glycosylation—G0 glycan, the 2AB glycan analysis showed variability from 2-6%. To set the target profile, the range is extended by 1% on the lower limit and 2% on the upper limit yielding a target profile range of 1%-8%. Using this method target is set for each modification.

TABLE 1
Setting Target Profile

| PTM | Batch 1 | Batch 2 | Batch 3 | Batch 4 | Batch 5 | Target Profile Range |
|---|---|---|---|---|---|---|
| Glycosylation -G0 | 3.5% | 2% | 5% | 6% | 3% | 1-8% |
| Glycosylation-G1 | 1.5% | 2% | 1.8% | 2.5% | 0.5% | 0-4.5% |
| Glycosylation- G2 | 0% | 0% | 0% | 0% | 0% | 0% |
| Glycosylation -G0F | 45% | 48% | 51% | 44% | 52% | 44-54% |
| Glycosylation- G1F | 20% | 22% | 18% | 16% | 20% | 15-24% |
| Glycosylation- G2F | 4% | 3% | 5% | 4.5% | 6% | 2-8% |
| Glycosylation- | 1.5% | 1.8% | 1.7% | 1.6% | 1.9% | 0.5-3.9% |
| Mannose 5 | | | | | | |
| Glycosylation- | 0% | 0% | 0% | 0% | 0% | 0% |
| Mannose 8 | | | | | | |
| C-terminal lysine | 0.5% | 0.8% | 1% | 1.4% | 1.3% | 0-3.3% |
| content- 2 lysines | | | | | | |
| C-terminal lysine | 5% | 4% | 3% | 2% | 4% | 2-7% |
| content- 1 lysine | | | | | | |
| Deamidation | 3% | 3.5% | 3.2% | 4% | 3.5% | 2-6% |
| Oxidation | 2% | 2.5% | 2.1% | 1.8% | 3% | 0.8-5% |
| Aggregation | 0.5% | 0.4% | 0.5% | 0.4% | 0.3% | 0-2.5% |

### EXAMPLE 2

### A Recipe for Biosimilar of Herceptin® with A Similar Glycosylation

This example demonstrates one method to obtain a recipe for making a biosimilar of Herceptin® focusing on optimization of the glycosylation pattern. Herceptin® (INN: Trastuzumab) is a humanized monoclonal antibody directed against the external domain of the human HER2. The antibody is an IgG1, consisting of two γ₁ heavy chains, two κ chains, and a single complex-type biantennary N-linked glycan at Asn300 of the heavy chain. For the purpose of this example Herceptin® (INN: trastuzumab) is a reference product. Five different batches of Herceptin® were analyzed for glycosylation pattern using 2AB glycan labeling method and the results are shown in Table 2. Since the modification identity for some chromatography peaks remains unknown, not all peaks could be assigned to specific modifications. Therefore, modifications have been labeled using peak numbers. An example of a chromatogram showing the glycan peaks representing different modifications from the 2AB glycan method with labeled peaks is shown in FIG. 3. To set target profile, the measurements for each modification for 5 batches of Herceptin® were first collected. For example for Peak 1 modification the range was shown to be 1.7-2.8%. Based on the method shown in Example 1. the target profile was identified to be 0.7-4.8% (lower limit was extended by 1% and upper limit was extended by 2%).

TABLE 2
Setting Target Profile For Glycan Species on Herceptin ®

| Glycan Species | H4103 | H0783 | H0790 | H0792 | 911826 | Target Profile Range |
|---|---|---|---|---|---|---|
| Peak 1 | 2.3 | 2.8 | 2.2 | 2.0 | 1.7 | 0.7-4.8% |
| Peak 2-G0 | 3.6 | 3.2 | 3.3 | 3.8 | 3.6 | 2.2-5.6% |
| Peak 3 | 1.7 | 1.8 | 1.8 | 1.5 | 3.5 | 0.5-5.5% |
| Peak 4- G0F | 45 | 49 | 47 | 45 | 45 | 44-51% |
| Peak 5 | 1.6 | 2.0 | 1.9 | 2.0 | 0.7 | 0-4% |
| Peak 6-G1 | 1.2 | 1.0 | 1.1 | 1.3 | 1.0 | 0-3.2% |
| Peak-7-G1F/mannose 5 | 0.9 | 0.9 | 1.0 | 0.0 | 1.2 | 0-3.2% |
| Peak 8-(1,6)G1F | 25 | 22 | 24 | 26 | 23 | 21-28% |
| Peak 9 (1,3)G1F | 10.3 | 10.1 | 10.6 | 10.4 | 10.6 | 9.1-12.6% |
| Peak 10 G2F | 5.6 | 4.4 | 4.9 | 5.2 | 6.1 | 3.4-8.1% |
| Peak 11 | 0.9 | 0.8 | 1.0 | 1.2 | 0.6 | 0-3.2% |
| Peak 12 | 0.3 | 0.4 | 0.4 | 0.4 | 0.3 | 0-2.4% |
| Peak 13 | 0.3 | 0.4 | 0.4 | 0.4 | 0.4 | 0-2.4% |
| Peak 14 | 0.7 | 0.8 | 0.8 | 0.9 | 1.0 | 0-3.0% |
| Peak 15 | 0.3 | 0.5 | 0.5 | 0.5 | 0.7 | 0-2.7% |

To obtain a recipe for production of a biosimilar with a similar glycosylation pattern to the original Herceptin®, CHO cells engineered to express the recombinant protein with an amino acid sequence identical to trastuzumab were first grown in the growth media without any inhibitors to establish a baseline. The glycan Species were analyzed using 2AB glycan method. The data generated for the Baseline is shown in Table 3. It was observed that Peak 2 (G0) and Peak 6 (G1), and Peak 7 (mannose-5 and G1′) modifications were lower for the biosimilar than their target profile.

G0, G1 and G1′ modifications are non-fucosylated modifications and are controlled by a host protein called fucosyl transferase and the mannose-5 modification is controlled by the host protein known as α-mannosidase 1. Fucosyl transferase can be inhibited by a variety of fucosyltransferase inhibitors shown in FIG. 2, α-mannosidase 1 can be inhibited by kifunensine.

The result of optimization is shown in Method 1 in Table 3. Briefly to obtain trastuzumab with modifications in the target range, cells were placed in growth media and treated with 2F-Peracetyl-Fucose (FTI) on day 7 at different concentrations (20 μM. 10 μM, 5 μM, 1 μM, 0.1 μM) to identify optimal drug concentration. On day 12 cells were harvested and the trastuzumab biosimilar isolated. 2AB glycan analysis of the biosimilar showed that while 20 μM FTI treatment resulted in an increase of G0, G1 and G1′ PTMs above that of target PTMs, 10 μM FTI treatment resulted in G0, G1 and G1′ levels that matched the target PTM range. When cells were treated with FTI at concentrations lower than 8 μM the modification were outside the target range. FTI concentrations used to reach target profile are cell specific so it is expected that different concentrations of the FTI or other modulators would be required when a starting cell line is different from the one described in this example.

Different treatment methods such as Method 2 can be used to obtain target profile. For example, FTI can be added on a daily basis starting on day 5 (Table 3, Method 2) rather than on Day 7. Treatment of cells expressing trastuzumab biosimilar with FTI at about 1.5-3.5 μM everyday starting on Day 5 produced similar results to the one time treatment on Day 7 described in Method 1. Based on these results, different treatment schedules of FTI (different methods) can be employed to obtain the same effect.

In addition to demonstrating that fucosyltransferase activity can be modulated, this Example also demonstrates modulation of the activity of α-mannosidase I using kifunensine in Method 3. Method 3 demonstrates optimization of the mannose species by addition of kifunensine. Different amounts of kifunensine (KFI) were added on day 7 ranging from about 0.001 ng/ml-100 ng/ml. The ideal concentration was identified as being between about 1-10 ng/ml treated on Day 7. Since mannose-5 modification is not an important contributor to the biological activity of trastuzumab, this modulator may, but doesn't have to be included, in the recipe depending on the growth media used.

TABLE 3
Methods for Modulating modifications on a Trastuzumab Biosimilar

| | Baseline- | Method 1 - | Method 2 - | Method 3 - 10 μM | |
|---|---|---|---|---|---|
| | Growth | 10 μM | 2.5 μM-3.5 μM | FTI and 5 ng/ml | |
| Glycan | media | FTI - | FTI every day | KFI on | Target profile |
| Species | only | Day 7 | starting at day 5 | Day 7 | range |
| Peak 1 | 1.5% | 1.5% | 1.5% | 1.5 | 0.7-4.8% |
| Peak 2-G0 | 1% | 4% | 4% | 4% | 2.2-5.6% |
| Peak 3 | 1.5% | 1.5% | 1.5% | 2% | 0.5-5.5% |
| Peak 4-G0F | 47% | 44% | 44% | 44% | 44-51% |
| Peak 5 | 0.8% | 0.8% | 0.8% | 0.8% | 0-4% |
| Peak 6-G1 | 0.6% | 1.8% | 1.8% | 1.6% | 0-3.2% |
| Peak 7- | 0.6% | 1.2% | 1.2% | 2% | 0-3.2% |
| G1F/mannose 5 | | | | | |
| Peak 8- | 26% | 26% | 26% | 25.5% | 21-28% |
| (1,6)G1F | | | | | |
| Peak 9 | 12% | 11% | 11% | 11% | 9.1-12.6% |
| (1,3)G1F | | | | | |
| Peak 10 G2F | 6% | 6.5% | 6.5% | 6% | 3.4-8.1% |
| Peak 11 | 0.2% | 0.2% | 0.2% | 0.2% | 0-3.2% |
| Peak 12 | 0.25% | 0.25% | 0.25% | 0.25% | 0-2.4% |
| Peak 13 | 0.2% | 0.2% | 0.2% | 0.2% | 0-2.4% |
| Peak 14 | 0.2% | 0.2% | 0.2% | 0.2% | 0-3.0% |
| Peak 15 | 0.2% | 0.2% | 0.2% | 0.2% | 0-2.7% |

### EXAMPLE 3

### Determining Recombinant Protein Variants and Their Biological Activity

This example describes a method for determining recombinant protein variants and their biological activity.

The difference between product modification and product variant is that product modifications can be measured and product variants cannot. A single or several product modifications can be measured at the same time depending on the analytical method used In the example below, there are two modifications on a recombinant protein product, modification 1 and 2. There are also other measurements that were made that provide additional information about the product, such as that 25% of the product is not modified as well as that 25% of the product contains two modifications. Based on this information, one skilled in the art can determine that the product, is a complex mixture of 4 product variants; product variant #1 contains 2 modifications and is present at 25% in a complex mixture, product variant #2, containing only modification 1, is present in the complex mixture at the abundance of 25%, product variant 3 is present at 25% and unmodified product variant #4 is also present at 25%.

Furthermore, the set of modifications on product variant #1 is modification 1 and 2, the set of modifications on product variant 2 is only one modification #1, the set of modifications on product variant #3 is modification 2; product variant 4 has no modifications.

The rationale for determining the type and the abundance of product variants and not modifications because it is the product variants, and not product modifications that exert the biological activity. The biological activity of the complex mixture is the sum of biological activities of each variant.

1. A method for producing a biosimilar product having fingerprint similarity lo a reference product comprising;
a. identifying at least one relationship between a modification in a reference product that is a biologic molecule and its biologic activity;
b. measuring the amount of the modification found on the reference product in more than one batch of the reference product using an analytical assay;
c. setting a target range for the amount of the modification in the reference product based on the measured amounts in b;
d. growing living cells expressing a recombinant protein that is a biosimilar molecule having the biological activity of the reference product;
e. isolating the biosimilar product trom d) and comparing its modification to the target range set in c;
f. selecting a plurality of growth media having one or more modulators that change the modification on the biosimilar molecule and growing the cells in the presence ot said modulators to produce more than one batch of the biosimilar product;
g. comparing the modifications of the batches of biosimilar products from f to the target range set in c;
h. repeating steps f) and g) with additional modulators and/or at different modulator concentrations until the biosimilar product matches the target range set in c to establish a protocol for the production of said biosimilar within the target range of the modification in c;
i. isolating the biosimilar product having the modification set for said reference product in c;
j. measuring the quantity of the modification on the isolated biosimilar product;
k. repeating steps f thru j until the isolated reference product hits an amount of the modification that is within 80 to 120 percent of the target range set in c.

2. The method for producing a biosimilar product having fingerprint similarity to a reference product of claim 43, farther comprising identifying more than one relationship between a modification in a reference product or biosimilar and its biologic activity.

3. The method for producing a biosimilar product having fingerprint similarity to a reference product of claim 43, wherein the target range for the amount of the modification in the reference product is from the lowest to the highest amount of the modification identified in b.

4. A method for identifying recombinant protein variants in a complex mixture comprising, measuring the amount of a modification in a recombinant protein product which is a complex mixture, determining the structure and abundance of all potential product variants, wherein each variant contains a different set of modifications constrained by the abundance of each modification in said complex mixture.

5. The method of claim 46 for identifying recombinant protein variants in a recombinant protein which is a complex mixture further comprising measuring several modifications in she complex mixture.

6. The method of claim 46 for identifying recombinant protein variants in a complex mixture wherein the product modifications are measured by size exclusion, ion exchange, reverse phase, hydrophobic interaction chromatography, intact and reduced mass.

7. The method of claim 46 for identifying recombinant protein variants in a complex mixture wherein the product modifications are measured by MS assays and include a peptide map and peptide map MS/MS.
